(19) United States
(12) Patent Application Publication
(10) Pub. No.: US 2019/0328224 A1
(43) Pub. Date: Oct. 31, 2019

(54) VIDEO LARYNGOSCOPE ACCESSORY FOR A MOBILE COMMUNICATION DEVICE

(71) Applicant: Nicholas Pearse NEVIN, Cape Town (ZA)

(72) Inventor: Nicholas Pearse NEVIN, Cape Town (ZA)

(21) Appl. No.: 16/473,783

(22) PCT Filed: Dec. 20, 2017
(86) PCT No.: PCT/IB2017/058205

§ 371 Date: Jun. 26, 2019

(30) Foreign Application Priority Data
Jan. 3, 2017 (ZA) 2017/00001
Sep. 13, 2017 (ZA) 2017/06207

(51) Int. Cl. A61B 1/267; H04M 1/04; A61B 1/05; A61B 1/015

(52) U.S. Cl. CPC A61B 1/267; A61B 1/015; A61B 1/05; H04M 1/04

(57) ABSTRACT

An accessory (10) is provided for using a mobile phone (56) as part of a video laryngoscope. The accessory (10) has a body (12) that defines a laryngoscope blade (14) and a holder (18) in which the mobile phone (56) is held. The blade (14) has a camera (28) at its distal end (16) and electronic circuitry that receives image signals from the camera (28) and transmits them to be displayed on the mobile phone (56). The laryngoscope body (12) defines a handle (66) that is attached to the proximal end (68) of the blade (14) and the blade (14) has a curved shape between its proximal and distal ends (68,16) that curves through an angle of more than 90 degrees, preferably about 110 degrees.

### FIELD OF THE INVENTION

This invention relates to video laryngoscopy and in particular, the use of a mobile communication device for display during video laryngoscopy, as well as the incorporation of in-line suctioning or apnoeic oxygenation capability into the device itself.

### BACKGROUND TO THE INVENTION

Video laryngoscopy is widely used when intubating patients, especially in the management of a difficult airway, but the equipment required for video laryngoscopy is usually very costly and the cost of monitors in video laryngoscopes typically contributes very substantially to the overall cost.

The cavity through which a video laryngoscope has to be passed (the oral pharynx) is often prone to accumulation of liquids such as blood, saliva and vomitus, especially in trauma situations and the liquids tend to obstruct the laryngoscope's lens. If suction is available, the blade of the laryngoscope is either withdrawn temporarily while suction is applied to withdraw the liquid, after which the blade is reinserted, or suctioning is applied under direct vision of the blade. Either way the patient cannot be intubated during this process. These scenarios are time consuming, typically require two operators, increases the risk of injury during re-placement of the blade and are often ineffective if the rate of liquid accumulation is too rapid. It also delays the placement of a tube, which in turn delays oxygenation and ventilation of the patient.

When intubating a patient using a laryngoscope, the operator needs to manipulate the laryngoscope and endotracheal tube by hand and these movements can cause injury to the patient. The risk of incorrect manipulation and injury is especially high in emergencies, where operators are often not assisted and need to work quickly. The risk of injury is also increased if the operator has to look in different directions to attend to the tasks of watching the patient and the video feed from the laryngoscope, operating suction and intubating the patient.

Due to the cost of video laryngoscopes, the complexity of operating them and the need for assistance, video laryngoscopy is not used as widely is it could be, especially in emergencies and especially in communities with limited resources.

Placement of a video laryngoscope and endotracheal intubation both hold significant risks of causing injury to a patient's upper airway, especially if more than one attempt at intubation is required. The risks are also increased in patients with difficult airways (e.g. an anterior larynx) and these risks persist despite the existence of various competing designers and suppliers of video laryngoscopes. In addition, the mitigation of these risks through the existing devices has still not yet been successfully addressed, despite being the subject of numerous discussions, studies, and the like.

The present invention seeks to provide a video laryngoscope that is cost-effective, easy to use and addresses the challenges mentioned above, at least in part.

### SUMMARY OF THE INVENTION

According to a first aspect of the present invention there is provided an accessory for a mobile communications device, said accessory comprising a body defining a laryngoscope blade and a holder that is configured to receive a mobile communication device, said blade having a distal end and including a camera in the proximity of the distal end, and electronic circuitry that is configured to receive an image signal from the camera and to provide a display signal to the mobile communication device.

The word “image” is used herein to refer broadly to visually perceptible images and it predominantly refers to video, but can also be interpreted to refer to “still” images—bearing in mind that video is merely a rapid succession of still images.

The holder may be configured for detachable attachment of the mobile communication device to the body. E.g. the holder may comprise a recess in which at least part of the mobile communication device is receivable and/or one or more clamp elements that can grip the mobile communication device. In a preferred embodiment, the holder may comprise a recess in which an edge of the mobile communication device is receivable and a clamp that is biased to urge the edge of the mobile communication device into the recess.

The blade may define a suction inlet in the proximity of the distal end and a suction passage that is in communication with the distal end and that extends proximally to a suction outlet. The accessory may include a suction controller such as a valve that can open and close the suction passage.

According to another aspect of the present invention there is provided a laryngoscope comprising a body defining a handle and a blade, the handle being attachable to the proximal end of the blade and the blade having a distal end, said blade having a curved shape between its proximal and distal ends, and said body being curved through an angle of more than 90 degrees between a longitudinal axis of the handle and a longitudinal axis of the distal end of the blade.

Preferably, the body of the laryngoscope curves through an angle of about 110 degrees between the longitudinal axes of the handle and the distal end of the blade.

The handle may be detachable from the proximal end of the blade or may be integrally formed with the blade and the handle may have an orientation that is offset from the proximal end of the blade by an acute angle, preferably by about 20 degrees.

The blade may define a spatula at its distal end.

### BRIEF DESCRIPTION OF THE DRAWINGS

For a better understanding of the present invention, and to show how it may be put into effect, the invention will now be described by way of non-limiting example, with reference to the accompanying drawings in which:

FIG. 1 shows a distal three-dimensional view of a laryngoscope accessory for a mobile communication device according to the present invention;

FIG. 2 shows a proximal three-dimensional view of the laryngoscope accessory of FIG. 1 from the right;

FIG. 3 shows a proximal three-dimensional view of the laryngoscope accessory of FIG. 1 from the left;

FIG. 4 shows a front view of the laryngoscope accessory of FIG. 1;

FIG. 5 shows a left side view of the laryngoscope accessory of FIG. 1;

FIG. 6 shows a detail three-dimensional rear view of a holder of the laryngoscope accessory of FIG. 1;

FIG. 7 shows a sectional side view of the larynx of a patient, with the laryngoscope accessory of FIG. 1, in use; and

FIG. 8 shows a right side view of the laryngoscope accessory of FIG. 1.

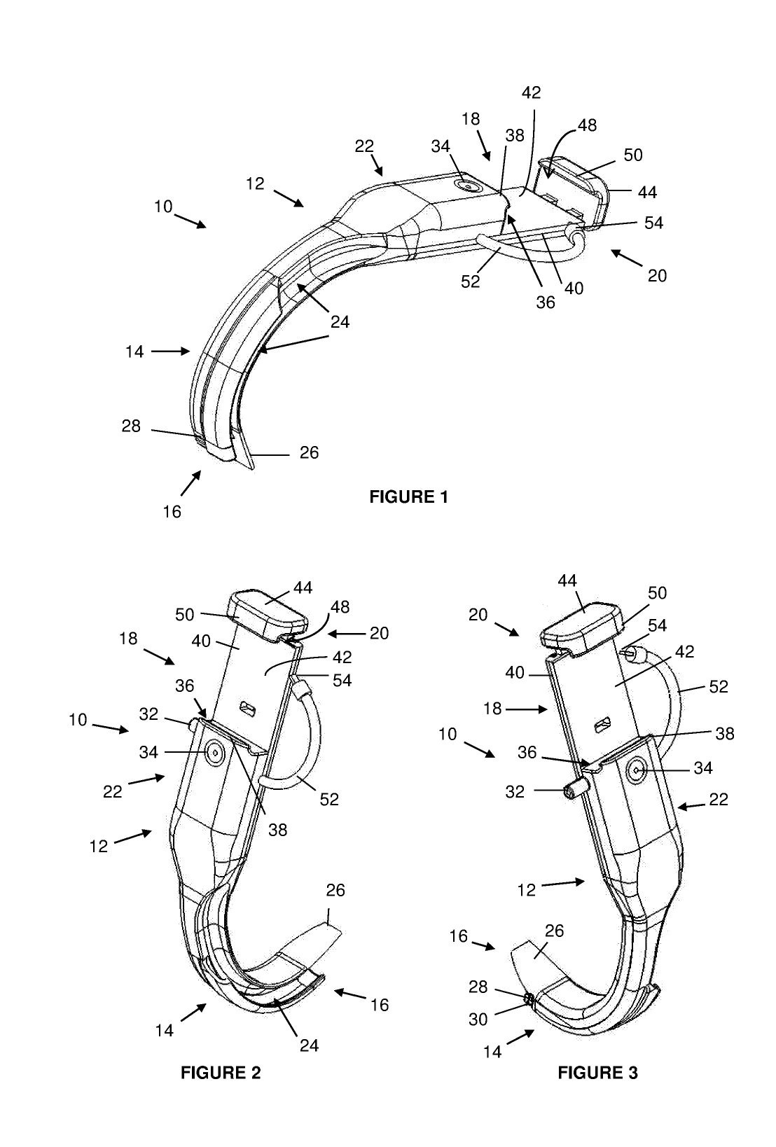

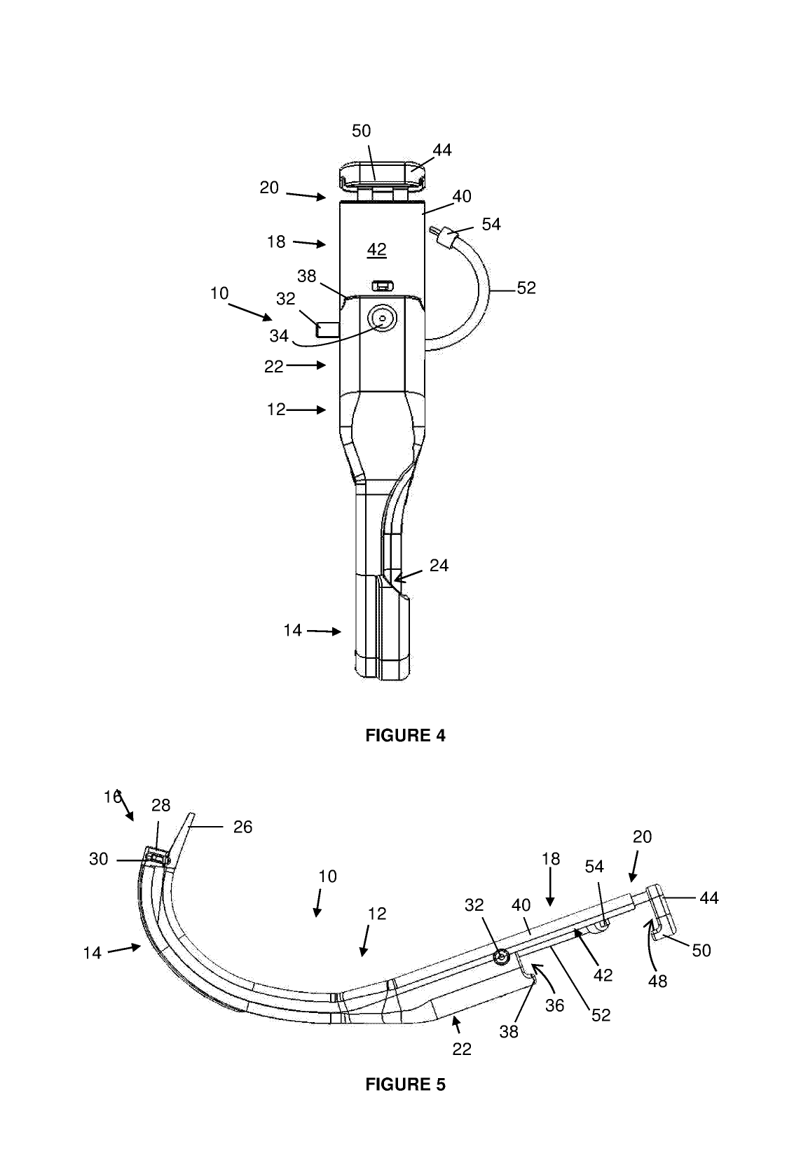

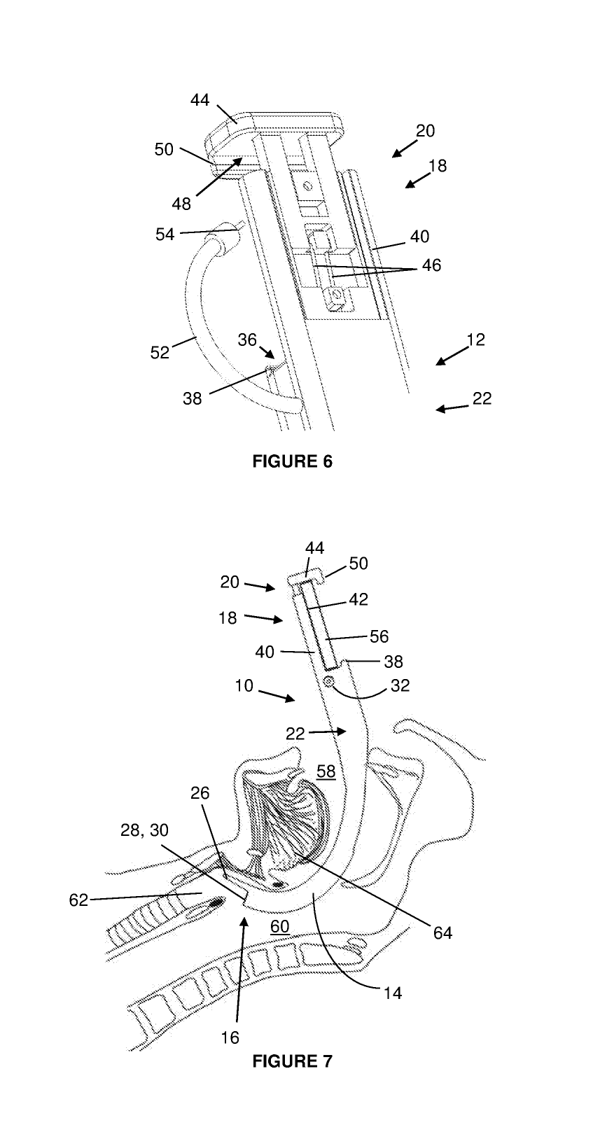

### DETAILED DESCRIPTION OF THE DRAWINGS

Referring to the drawings, a laryngoscope accessory according to the present invention, is generally identified by reference number 10 and is referred to herein below as “accessory”, for brevity.

The main part of the accessory 10 is a body 12 that can be a unitary component or an assembly and that can be made from materials that can be washed for re-use. The options for manufacture of the body 12 are varied, but the body must be made of a material that can be sterilised and that is sufficiently durable for re-use to be possible. However it is possible for the accessory to be for a single use. The material of the body 12 is preferably light in weight and low in cost and in a preferred embodiment, the body is an assembly of moulded plastics components.

The body 12 forms a laryngoscope blade 14 (referred to as “blade” herein below, for brevity) at a distal end 16 of the body and forms a holder 18 at a proximal end 20 of the body. In the preferred embodiment, the body 12 also forms an intermediate part 22 between the blade 14 and the holder 18.

The blade 14 has a curved shape and it defines a lateral passage 24 along which a tube can be passed, to intubate a patient. At the distal end 16, the blade 14 forms a tongue spatula 26 and the distal end of the lateral passage 24 is recessed below the spatula. The blade 14 includes a camera 28 that is also recessed below the spatula 26, adjacent the distal opening of the lateral passage 24 and a suction inlet opening 30 is defined adjacent the camera. In the illustrated embodiment, the inlet opening 30 is disposed laterally on the blade 14, but in another preferred embodiment, the inlet opening can instead be disposed on the underside of the blade, at its distal end 16.

The body 12 defines an internal suction passage that extends in a proximal direction from the suction inlet opening 30 to a suction outlet opening 32 on the side of the intermediate part 22 and the outlet opening is preferably configured to be attached to a conventional suction device. Inside the body 12, a suction controller is provided to open and close the suction passage selectively and the suction is controlled by occluding the suction button 34 on the front of the intermediate part 22.

The holder 18 comprises a recess 36 formed at a proximal end of the intermediate part 22, between a lip 38 at the front, and a back support 40. The back support 40 extends in a proximal direction and defines a face 42 at its front. At the proximal end 20, the accessory 10 includes a clamp element 44 that is supported by the back support 40 and that can slide longitudinally relative to the back support, i.e. it can slide along the back support, towards and away from the recess 36. The clamp element 44 is connected to the back support 40 with a bias element in the form of an elastic band 46. Various other bias elements can be used, but the elastic band 46 is particularly cost-effective. The clamp element 44 also defines a recess 48 behind a lip 50.

Electronic circuitry is provided inside the body 12 that is configured to receive an image signal from the camera and to transmit a display signal. These electronic functions can be performed in various ways, but in a preferred embodiment, the image signal and the display signals are digital electronic signals and the display signal is transmitted from the circuitry via a cable 52 that extends out of the body 12 to a conventional connector such as a micro USB connector 54. The circuitry is configured to transmit the display signal in a format that allows it to be used in a conventional mobile communications device such as a smartphone.

Various features have been described that are internal to the body 12 and the intermediate part 22 partly serves as a housing for these parts, but also serves as a handle for an operator to hold and manipulate the accessory 10.

To use the accessory 10, a mobile communication device such as a mobile telephone, preferably a smartphone 56 is attached to the body 12 with the holder 18. (Other communication devices such as tablet computers, or the like can also be used.) This is done by inserting one edge of the smartphone 56 in the recess 36 with a screen of the smartphone facing forwards, pulling the clamp element 44 away from the back support 40 (against the bias of the elastic band), resting the back of the smartphone against the face 42 and releasing the clamp element. Tension in the elastic band 46 urges the clamp element 44 towards the back support 40 so that an edge of the smartphone 46 is received in the recess 48 and the clamp element urges the opposite edge into the recess 36. The smartphone 56 is thus firmly held captive with two opposing edges received in the recesses 36,48 and with its screen facing forwards.

The accessory 10 is connected to the smartphone 56 by inserting the connector 54 in a socket on the smartphone so that the image signal is supplied to the smartphone. Suitable software is installed on the smartphone 56, if necessary, and the smartphone converts the image signal to visual images that are displayed on its screen—i.e. to video.

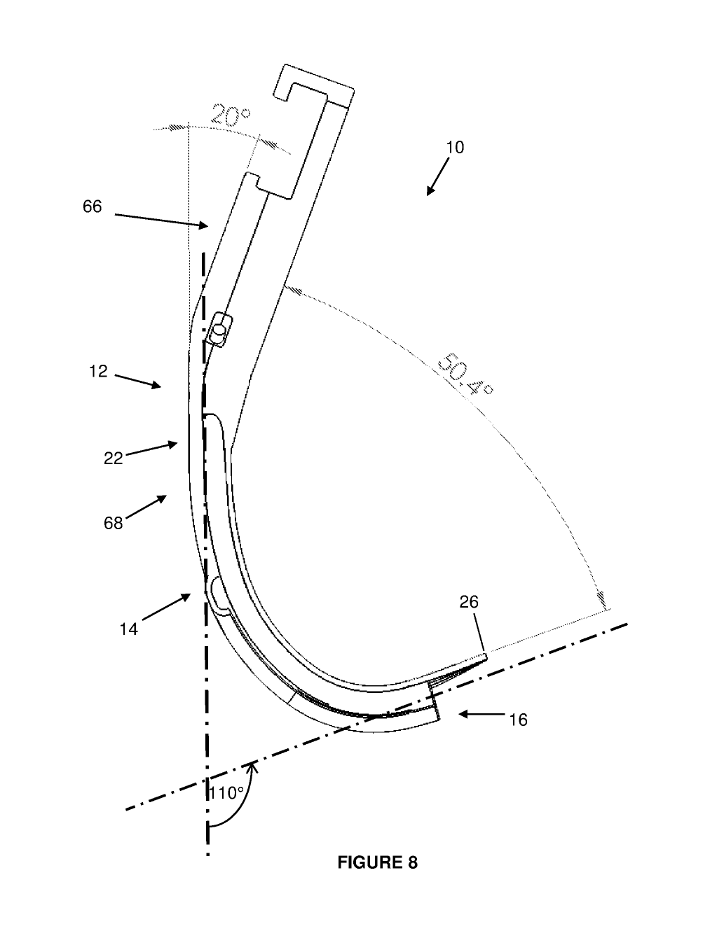

Referring in particular to FIG. 8, the part of the holder 18 that is immediately adjacent the intermediate part 22, forms a handle 66 where the accessory 10 can be held comfortably. The end of the blade 14 that is immediately adjacent the intermediate part 22 forms a proximate end 68 of the blade. The whole body 12 has an elongate, curved shape and each part of the body has a longitudinal axis, although these longitudinal axes are not shown in the drawings, apart from FIG. 8.

The handle 66 is orientated with a small offset angle relative to the proximal end 68 of the blade 14. This offset angle could be varied, but should preferably be an acute angle, preferably about 20 degrees. This offset angle is shown in FIG. 8 between the outer surfaces of the handle 66 and the proximal end 68 of the blade, but this is also the offset angle between the longitudinal axes of these parts.

The blade 14 is curved between its proximal end 68 and the distal end 16 of the accessory (which is also the distal end of the blade) and this curved shape is such that the distal end 16 of the blade is oriented at an angle of 50 degrees relative to the handle 66. The angle of 50 degrees is shown in FIG. 8 between the spatula 26 and the outer surface of the handle 66, but the angle is the same, if measured between longitudinal axes of the handle and blade 14.

The combined effect of the offset angle of 20 degrees between the handle 66 and the proximal end 68 of the blade 14 and the curvature of the blade so that its distal end 16 is oriented 50 degrees relative to the handle, is that distal end is oriented at an angle of 70 degrees relative to the proximal end of the blade, or that the blade is curved through an angle of more than 90 degrees, preferably about 110 degrees, between its proximal and distal ends. This angle is shown in FIG. 8.

In use, the accessory 10 (with the smartphone 56) is used to intubate a patient as shown in FIG. 7, by inserting the blade 14 into the patient's mouth 58 and pharynx 60 to reach the larynx 62, around the tongue 64. While placing the blade 14, the operator can grip the accessory 10 by hand around the handle 66 and can manipulate it, while watching the images captured by the camera 28 and displayed on the screen of the smartphone 56. If required, the operator can operate the suction button 34 with the thumb of the hand holding the intermediate part 22, to withdraw liquids via the suction inlet 30 in the vicinity of the camera 28. When the operator is satisfied with the placement of the blade 14, a tube can be fed along the lateral passage 24 to intubate the patient.

The operator only needs one hand to manipulate the accessory 10 and operate the suction button 34 and can use the other hand to intubate the patient. Accordingly, the accessory can be used without an assistant and suction or the supply of oxygen can be applied with the blade 14 in place (i.e. without the need to withdraw the blade temporarily to apply suction).

Furthermore, the screen of the smartphone 56 is in the proximity of the patient's mouth, where the accessory 10 is manipulated and where the intubation is performed. The operator can thus have the patient, the accessory 10, the suction button 34 and the tube in this field of vision, while watching the video display on the smartphone's screen.

The monitors used in conventional video laryngoscopes contribute substantially to the cost of the laryngoscopes and the use of a smartphone 56 according to the present invention avoids the cost of a monitor. In addition, the use of a smartphone provides functionality that is already built into most smartphones, such as video recording, still image capturing, feeding still or video images, and the like. A stand-alone smartphone application facilitates all features of video and still image capturing, as well as storage and external communication of images, in a single platform. The features of the application allow an operator to transmit the video image captured by the camera 28 in real time, e.g. to a remote advisor and this can be of enormous assistance to a clinician using the accessory, who needs advice, as well as to display the image to several external individuals for the purpose of teaching. The video images may be stored in a cloud server, with appropriate encryption, for medico-legal risk mitigation as well as research.

Some advantages of the present invention are apparent from the description above, but the applicant believes that the accessory 10 can be produced at sufficiently low cost, smartphones are in sufficiently wide use and the accessory can be operated with sufficient ease, that the invention will enable many clinicians to use video laryngoscopy, who previously did not have access thereto, especially emergency service providers and clinicians in communities with limited resources.

The curved shape of the blade 14, curving through more than 90 degrees and preferably through about 110 degrees, allows the blade to be placed surprisingly easily, compared to known video laryngoscopes, thus reducing the risks of injuries to the upper airway.

1-10. (canceled)

11. An accessory for a portable consumer device, said accessory comprising:
a body defining a laryngoscope blade having a distal end forming a spatula;
a holder configured to receive the portable consumer device in the form of a mobile communications device;
a camera in a proximity of the spatula; and
electronic circuitry that is configured to receive a digital electronic image signal from the camera and to transmit a digital electronic display signal to the mobile communication device.

12. The accessory according to claim 11, wherein the holder is configured for detachable attachment of the mobile communication device to the body.

13. The accessory according to claim 12, wherein the holder comprises a recess in which at least part of the mobile communication device is receivable.

14. The accessory according to claim 13, wherein an edge of the mobile communication device is receivable in the recess and the accessory comprises a clamp element that is biased to urge the edge of the mobile communication device into the recess.

15. The accessory according to claim 12, wherein the holder comprises one or more clamp elements configured to grip the mobile communication device.

16. The accessory according to claim 11, comprising a suction passage that is in communication with the distal end and that extends proximally to a suction outlet, said suction passage being defined by the body and wherein the blade defines a suction inlet in a proximity of the distal end

17. The accessory according to claim 16, wherein said accessory includes a suction controller that is configured to open and close the suction passage.

18. A laryngoscope comprising a body defining a handle and a blade, the handle being attachable to a proximal end of the blade and the blade having a distal end, said blade having a curved shape between the proximal and distal ends, wherein the blade curves through an angle of about 110 degrees between the proximal and distal ends of the blade and the handle has an orientation that is offset from the proximal end of the blade by about 20 degrees, and the distal end of the blade is oriented at an angle of about 50 degrees relative to the handle.

19. The laryngoscope according to claim 18, wherein the handle is detachable from the proximal end of the blade.

20. The laryngoscope according to claim 18, wherein the blade defines a spatula at the distal end.
